(19) United States
(12) Patent Application Publication
(10) Pub. No.: US 2010/0233754 A1
(43) Pub. Date: Sep. 16, 2010

(54) Vessel Transporting Apparatus and Method

(76) Inventor: Christophe Alain Guex, South Maroubra (AU)

Correspondence Address: MAYBACK & HOFFMAN, P.A., 5722 S. FLAMINGO ROAD #232, FORT LAUDERDALE, FL, 33330, US

(21) Appl. No.: 12/673,705

(22) PCT Filed: Jul. 28, 2008
(86) PCT No.: PCT/AU08/01078

§ 371 Date: Feb. 16, 2010

(30) Foreign Application Priority Data
Aug. 15, 2007 (AU) 2007904368

(51) Int. Cl. C12Q 1/02; C12M 1/24

(52) U.S. Cl. 435/29; 435/307.1; 435/287.3

(57) ABSTRACT

This disclosure relates to an apparatus for transporting a vessel and to a sorting apparatus. The apparatus has at least one support device adapted to support the vessel in a reclined orientation, and a pushing mechanism adapted to transport the vessel along the support device. Advantageously, the apparatus is relatively simple and does not require vessels to contact an endless conveyor. Thus, vessels can be more readily rotated, weighed, etc. This disclosure also relates to a method of sorting vessels. One application of the apparatus and method is in the sorting of test tubes containing blood samples for testing.

### CROSS-REFERENCE TO RELATED APPLICATIONS

This application claims the priority, under 35 U.S.C. §§119, 120, 363, and 371 of Australian Patent Application No. 2007904368, filed Aug. 15, 2007, and International Application No. PCT/AU2008/001078, filed Jul. 28, 2008, which designated the United States, and was published in English, the entire disclosures of which are all hereby incorporated herein by reference in their entireties.

### STATEMENT REGARDING FEDERALLY SPONSORED RESEARCH OR DEVELOPMENT

n/a

### FIELD OF THE INVENTION

The present invention relates to apparatus and methods for transporting and/or sorting vessels, and more particularly to apparatus and methods for sorting test tubes containing blood samples for testing. However, it should be appreciated that the invention is not limited to this particular field of use. For example, the test tubes may contain samples of other biological substances, solids, jellies, or other liquids.

### BACKGROUND OF THE INVENTION

The diagnostic pathology departments of modern hospitals and diagnostic laboratories can receive thousands of test tubes containing patient's blood samples every day. The process of sorting these samples to direct them to the various tests and operations that each sample requires can be a time consuming and labor intensive task.

Typically, each test tube is labeled with a bar code that identifies the patient. The patient's records are normally stored in a database that is updated to include the tests and operations to be performed on each blood sample. In addition to testing, operations that may be required to be performed on each sample include aliquoting, centrifuging, addition of solutions or archiving.

Various apparatus for sorting test tubes are known. One such apparatus is the “RSD 800A” made by PVT Probenverteiltechnik GmbH. This apparatus requires an operator to initially load the test tubes into racks. An automated arm loads the test tubes one by one onto a conveyor which receives the test tubes in a vertical orientation. The test tubes travel along a conveyor, and each test tube can be selectively submitted for various processes, such as bar code labeling, aliquoting, decapping and recapping. Another automated arm removes each test tube from the conveyor and places it in an allocated test tube rack. This apparatus is noted for a number of inefficiencies. Firstly, the system requires initial manual loading and sorting. Secondly, the loading of test tubes in a vertical orientation can be slow. Thirdly, the speed of the conveyor is affected by the speed of the various processes. The PVT apparatus has a maximum throughput of 800 test tubes per hour. In practice, a significant number of test tubes do not need to undergo any aliquoting or decapping process. Hence, for such test tubes these processes are effectively redundant.

The “HCTS2000 MK2” made by m.u.t GmbH is an apparatus for sorting test tubes. The test tubes to be sorted are bulk loaded into a hopper and fed onto a conventional conveyor belt. The apparatus then transfers the test tubes to various output locations as required. The apparatus includes a bar code reader. Optionally, the apparatus can detect the test tube type and cap color. The disadvantage of this apparatus is that it uses conventional robotic arms, conveyor belts and carrousels to transport test tubes through the apparatus, which is expensive and complicated.

U.S. Pat. No. 7,141,213 B1 (Pang et al), U.S. Pat. No. 5,814,276 (Riggs), and Japanese Patent Abstract 04104058A (Aloka Co Ltd) also disclose various apparatus for processing test tubes. The sorting apparatus disclosed in these documents generally use complicated automation methods and require the test tubes to be loaded in an orderly manner, rather than loaded in bulk. U.S. Pat. No. 6,325,129 B1 (Wright et al) discloses an apparatus for sequentially transferring test tubes from a bulk loaded hopper.

The present invention seeks to provide a vessel transporting and/or sorting apparatus and method which will overcome or substantially ameliorate at least some of the deficiencies of the prior art, or to at least provide an alternative.

It is to be understood that, if any prior art information is referred to herein, such reference does not constitute an admission that the information forms a part of the common general knowledge in the art, in Australia or any other country.

### SUMMARY OF THE INVENTION

According to a first aspect of the present invention an apparatus for transporting a vessel is provided, comprising:

at least one support device adapted to support the vessel in a reclined orientation, and

a pushing mechanism adapted to transport the vessel along the support device.

Advantageously, the overall construction of the apparatus is relatively simple, making the apparatus economical to construct compared with other apparatus that use conventional robotics. Another advantage of this apparatus is that the vessels are not in contact with an endless conveyor and so can be more readily rotated, weighed etc. Thus, the vessels can be weighed without needing to be lifted.

Preferably, the apparatus further comprises at least two stations each station comprising a support device, wherein the pushing mechanism is adapted to transport the vessel from one station to another station.

Preferably, at least one of the stations is adapted to acquire information relating to the vessel and at least another of the stations is a transfer station adapted to transfer the vessel to one of two or more output locations depending on the information acquired, thereby sorting the vessel. It should be understood that acquiring information relating to the vessel may involve acquiring information about the vessel itself, the contents of the vessel, or both.

Advantageously, the throughput in vessels per hour of the apparatus is relatively high because each station is dedicated to only a single function, and the stations all operate simultaneously. The design of the transfer station in particular operates rapidly compared to most conventional robotic arms.

Preferably, at least one support device is rotatable to change the orientation of the vessel.

Preferably, the vessel is elongate and at least one support device is adapted to support the vessel in lengthwise alignment and the pushing mechanism is adapted to push the vessel lengthwise along the support device.

Preferably, the or each support device is adapted to support the vessel in lengthwise alignment.

Preferably, the apparatus further comprises a transducer adapted to determine the orientation of the vessel.

Preferably, the transducer comprises an arm that contacts the vessel as it is transported past the transducer.

Preferably, the orientation of the vessel is determined by measuring the position of the arm and the longitudinal position of the vessel as it is transported past the transducer.

Preferably, the arm of the transducer is mounted on a pivot.

Preferably, the transducer is adapted to measure the length of the vessel.

Preferably, the vessel has a round cross-section and the transducer is further adapted to measure the diameter of the vessel.

Preferably, the pushing mechanism comprises a plurality of protrusions, each protrusion being adapted to move between at least two stations and to contact the end of a vessel supported at one station and transport it to another station.

Preferably, the protrusions are attached to an endless conveyor in spaced apart relation.

Preferably, each support device has a recess extending along its length through which the protrusions pass in use.

Preferably, the recess in each support device is below a vessel supported by the support device in use.

Preferably, the support device of the transfer station is movable in a direction that is transverse to the main direction of travel of vessels through the station.

Preferably, the transfer station comprises a second pushing mechanism adapted to push a vessel out of the transfer station.

Preferably, at least one of the output locations is a vessel rack.

Preferably, at least one of the stations comprises a bar code reader adapted to read a bar code on a vessel.

Preferably, the vessel is elongate and the support device of the station comprising the bar code reader comprises a pair of rollers adapted to rotate a vessel on its longitudinal axis.

Preferably, the apparatus further comprises a database adapted to match the bar code of a vessel to a patient.

Preferably, the apparatus further comprises a feeder for feeding vessels to the at least one support device.

Preferably, the feeder comprises a hopper for receiving the vessels and a depositor for receiving the vessels from an outlet of the hopper and sequentially depositing them on at least one support device.

Advantageously, vessels can be poured directly into the hopper in any orientation, which saves considerable time and effort over prior art sorting apparatus that require the vessels to be loaded in an orderly manner, such as in racks.

Preferably, at least one of the stations is adapted to weigh a vessel. Advantageously, because the weight of the respective support device is known and the support device forms part of the scales, the vessel does not need to be lifted in order for its weight to be measured.

Preferably, at least one of the stations comprises an image capture device.

Preferably, the image capture device is adapted to detect the color of a cap of a vessel.

Preferably, the vessel is a test tube.

Preferably, the test tube is adapted to store a blood sample.

According to a second aspect of the present invention a method of sorting vessels is provided, comprising the following steps:
i. Depositing a vessel in a feeder,
ii. Feeding the vessel to a support device of a station where it is supported on its side in a reclined orientation, and
iii. Transporting the vessel, while in the reclined orientation, to another station.

Preferably, the vessel is a test tube.

According to a third aspect of the present invention, a sorting apparatus is provided for sorting a vessel, comprising:

at least one support device adapted to support the vessel in a reclined orientation, and

a pushing mechanism adapted to transport the vessel along the support device.

Preferably, the sorting apparatus further comprises at least two stations each station comprising a support device, wherein the pushing mechanism is adapted to transport the vessel from one station to another station.

Preferably, at least one of the stations is adapted to acquire information relating to the vessel and at least another of the stations is a transfer station adapted to transfer the vessel to one of two or more output locations depending on the information acquired, thereby sorting the vessel.

Preferably, the vessel is a test tube.

### BRIEF DESCRIPTION OF DRAWINGS

Notwithstanding any other forms which may fall within the scope of the present invention, a preferred embodiment of the invention will now be described, by way of example only, with reference to the accompanying drawings in which:

FIG. 1 shows a typical blood sample test tube.

FIG. 2 is a plan view of a test tube sorting apparatus in accordance with a preferred embodiment of the present invention.

FIG. 3 is a side view of the test tube sorting apparatus of FIG. 2.

FIG. 4 is a plan view of a feeder mechanism and bar code reader station of the test tube sorting apparatus of FIG. 2.

FIG. 5 is a partial sectional side view along V-V of the feeder mechanism and the bar code reader station of FIG. 4.

FIG. 6 is a sectional end view along VI-VI of the feeder mechanism of FIG. 5.

FIG. 7 is a plan view of the orientation, weighing and imaging stations of the test tube sorting device of FIG. 2.

FIG. 8 is a side view of the orientation, weighing and imaging stations of FIG. 7.

FIG. 9 is a partial sectional side view along IX-IX of the bar code reader station of FIG. 4.

FIG. 10 is an end view along X-X of the orientation station of FIG. 8.

FIG. 11 is a plan view of the transfer station of the test tube sorting apparatus of FIG. 2.

FIG. 12 is a side view of the transfer station of FIG. 11.

FIG. 13 is a sectional view along XIII-XIII of the transfer station of FIG. 11.

FIG. 14 shows the transfer station of FIG. 11 in a position to deposit a test tube into a container.

FIG. 15 shows the transfer station of FIG. 11 in a position to deposit a test tube into a two dimensional test tube rack.

FIG. 16 is sectional view along XVI-XVI of the transfer station in the position of FIG. 15.

FIG. 17 is a side view of an orientation station having an alternative support device for a test tube sorting apparatus in accordance with a preferred embodiment of the present invention.

FIG. 18 is an end view of the orientation station of FIG. 17.

FIGS. 19 and 20 are sectional side views through a device to transfer test tubes to a two dimensional test tube rack for a test tube sorting apparatus in accordance with a preferred embodiment of the present invention.

FIG. 21 is an end view of an orientation station having a support device for a test tube sorting apparatus in accordance with a preferred embodiment of the present invention.

### DETAILED DESCRIPTION OF THE INVENTION

The present invention may be embodied in a number of different forms and may be used for a number of different purposes. In a basic form, the invention is used for transporting vessels (e.g., test tubes) whilst in other forms (e.g., as depicted in the figures) it may be used for both transporting and sorting vessels (e.g., test tubes) and/or archiving vessels (e.g., archiving test tubes in fridges).

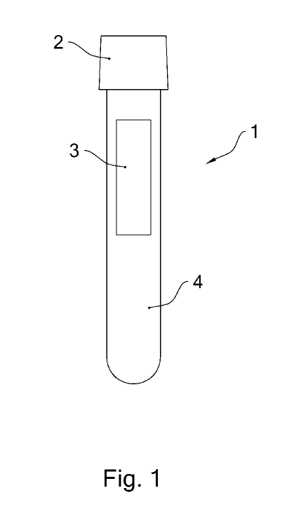

FIG. 1 shows a typical test tube 1 containing a blood sample. The test tube 1 has a cap 2 and a hollow body 4. A bar code 3 is affixed to the hollow body 4 and identifies the patient from whom the blood sample was taken.

The color of the cap 2 and the length and diameter of individual test tubes 1 may vary depending on the type of test tube. These parameters can be used to determine the tests or operations that are required to be performed.

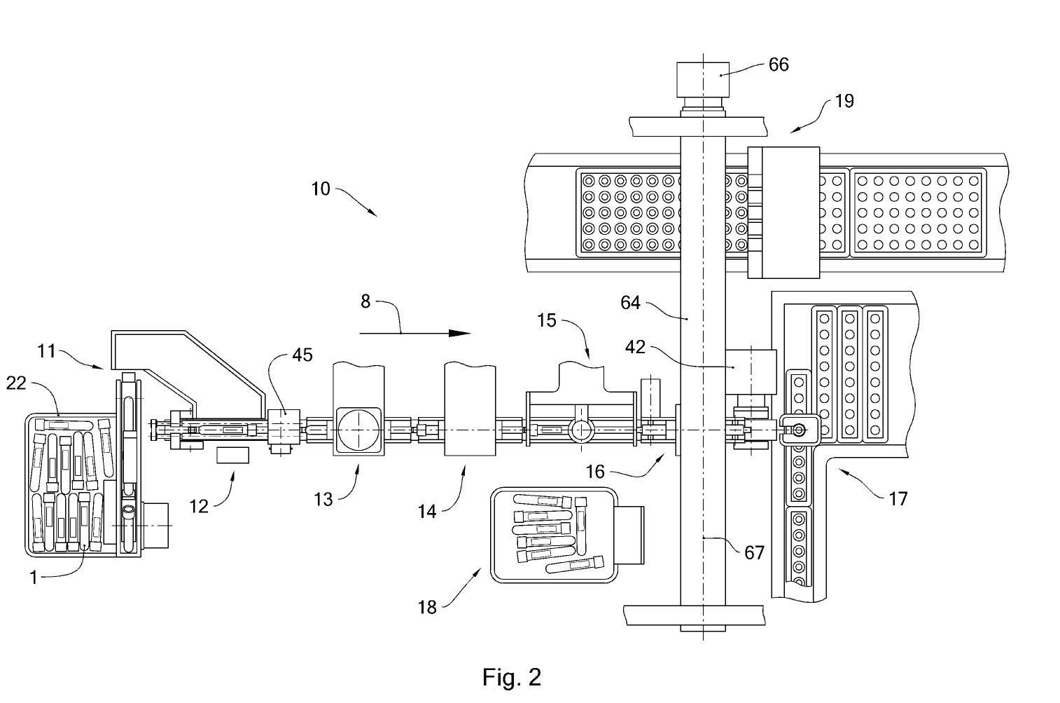

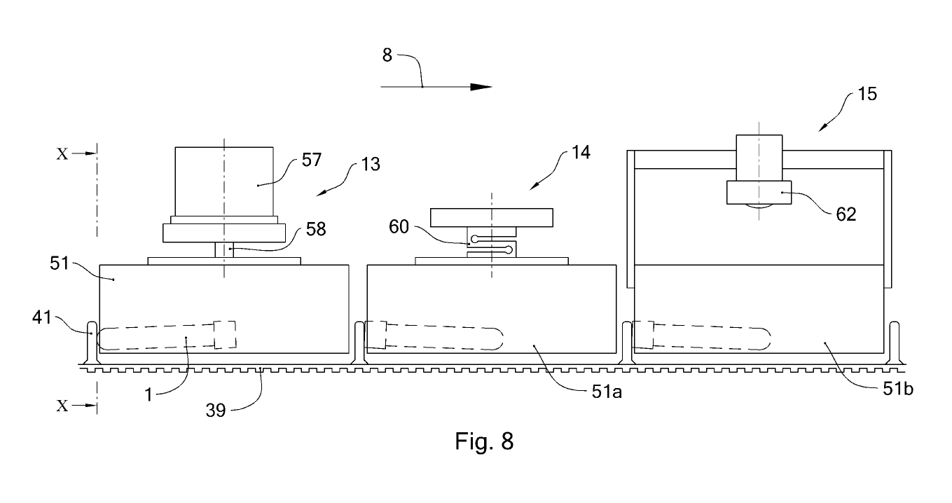

FIGS. 2 to 16 show a test tube transporting and sorting apparatus 10 (henceforth “sorting apparatus 10”) in accordance with a preferred embodiment of the present invention. For clarity, some components of the apparatus 10 are not shown (e.g. various actuators, bearings, sensors, support structures, etc that are commonly used in machine design).

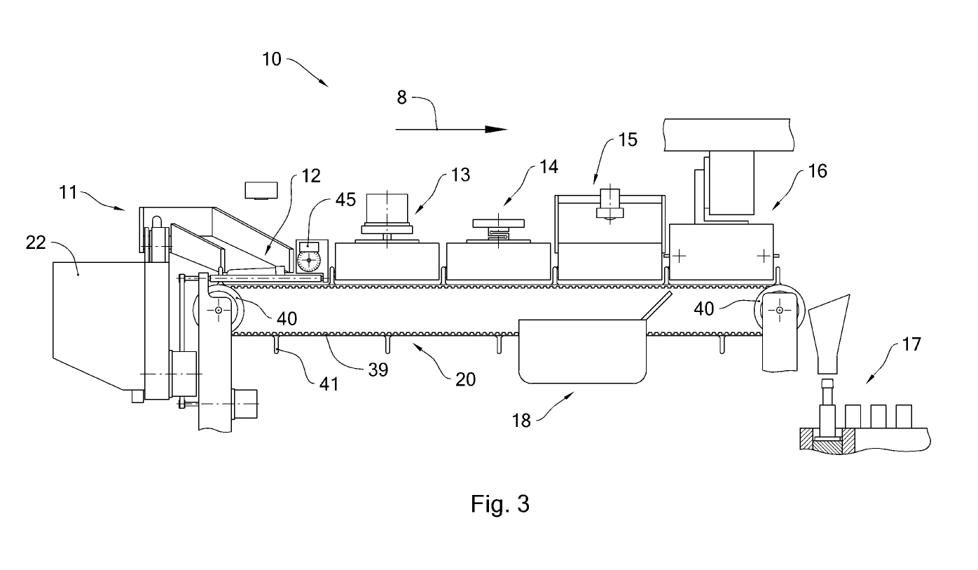

Referring to FIGS. 2 and 3, test tube sorting apparatus 10 comprises a hopper 22, a feeder mechanism 11, a bar code reader station 12, an orientation station 13, a weighing station 14, an imaging station 15 and a transfer station 16. The bar code reader station 12, weighing station 14 and imaging station 15 all involve acquiring information about the test tube 1. It should be understood that acquiring information relating to the test tube 1 may involve acquiring information about the test tube 1 itself, the contents of the test tube 1, or both. A pushing mechanism 20 sequentially transports test tubes 1 through each of the stations 12, 13, 14, 15 and 16 in a direction of travel indicated by arrow 8. The apparatus 10 is arranged such that test tubes 1 at the stations 12 to 16 are substantially aligned lengthwise. The hopper 22 receives bulk loaded test tubes 1 to be sorted. Test tubes 1 can be poured directly into the hopper 22 in any orientation, which saves considerable time and effort over prior art sorting apparatus that require the test tubes to be loaded in an orderly manner, such as in racks.

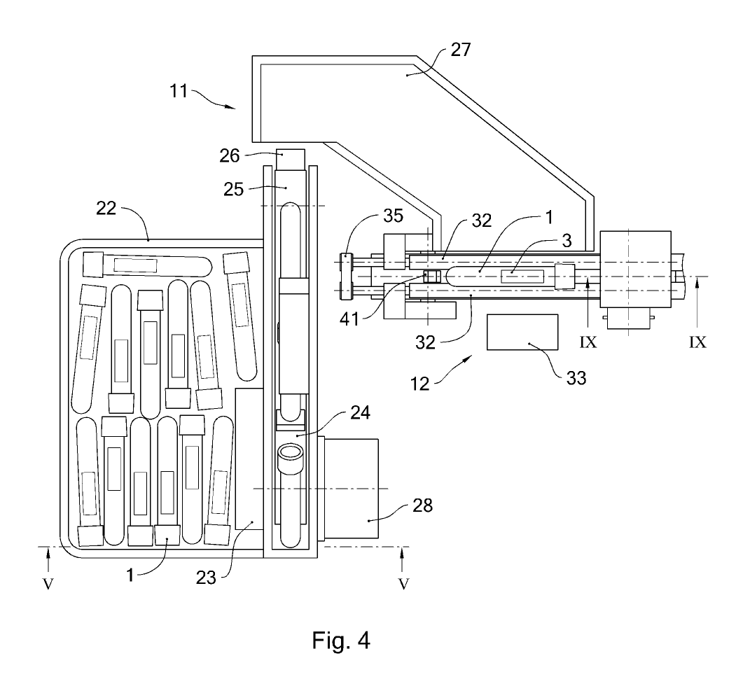

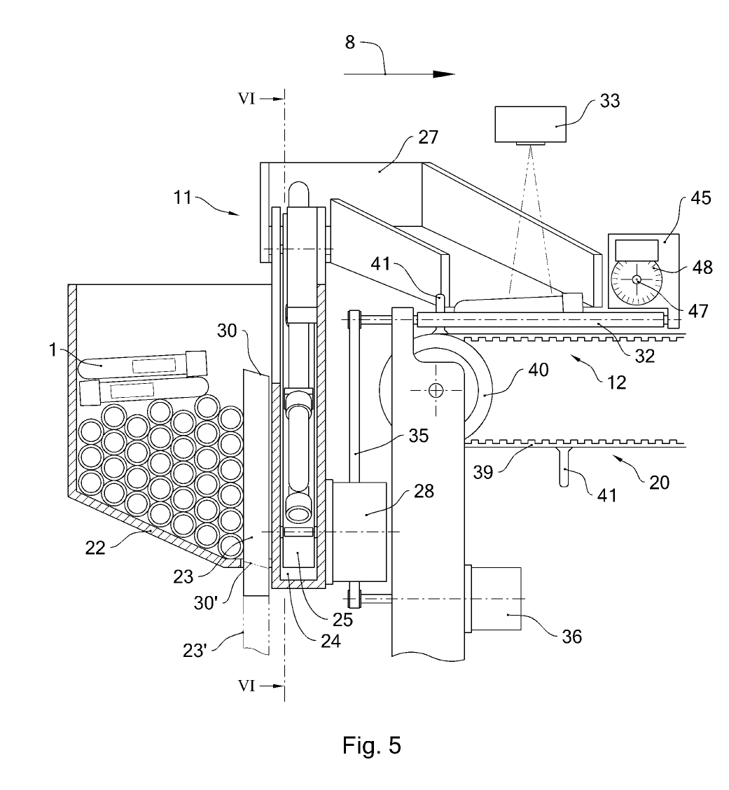

Referring to FIGS. 4, 5 and 6, the feeder mechanism 11 picks up test tubes 1 from the hopper 22 and sequentially deposits them at the bar code reader station 12, which in this embodiment is the first station of the sorting apparatus 10. As can be best seen in FIG. 5, the feeder mechanism 11 comprises a slide 23 that is raised and lowered inside the hopper 22 as required to transfer one or more test tubes 1 at a time to a secondary hopper 24. The slide 23 has a top 30 and is shown in its raised position. When the slide 23 is lowered, as indicated by dashed outline 23′, the top 30 of the slide 23 is at the bottom of the hopper 22, as indicated by dashed line 30′. This allows test tubes 1 in the hopper 22 to roll into the space vacated by the lowered slide 23. Then, as the slide 23 is raised, it lifts the test tubes 1 above it. The top 30 of the slide 23 is inclined such that test tubes 1 lifted by it roll off the top 30 of the slide 23 into the secondary hopper 24 when the slide 23 is raised to the position shown in which the top 30 of the slide 23 is just above an opening into the secondary hopper 24.

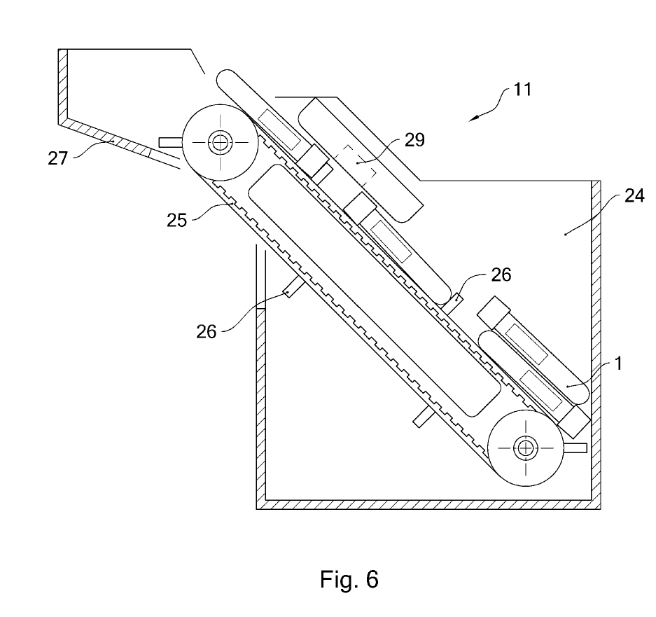

Referring to FIG. 6, an endless belt 25, with a plurality of spaced apart pegs 26 attached to it, carries test tubes 1 out of the secondary hopper 24 to a chute 27. A test tube 1 can travel up the belt 25 with either its cap 2 or body 4 facing forwards. The secondary hopper 24 provides only a few test tubes 1 at a time and so prevents the feeding mechanism 11 from jamming, which might otherwise occur if the endless belt 25 received test tubes directly from the main hopper 22.

When a test tube 1 is required to be deposited at the bar code reader station 12, a stepper motor 28 drives the belt 25 until a single test tube 1 is carried to the chute 27. This test tube 1 then rolls down the chute 27 onto two rollers 32 that form a support device for the bar code reader station 12. A sensor 29 detects the presence of a test tube 1 on the belt 25 and provides a signal to the stepper motor 28 to stop once the test tube 1 is delivered onto the chute 27. Test tubes 1 are deposited at the bar code reader station 12 in a random orientation, which means that either the cap 2 end or the tube end of the test tube 1 can face forwards in the direction of travel 8.

The bar code reader station 12 comprises the two rollers 32 and a bar code reader 33. The rollers 32 support the test tube 1 in a reclined orientation on its side, and are spaced apart such that there is an opening between them. The rollers 32 are rotated by a motor 36 through a belt 35. Rotating the rollers 32 rotates the supported test tube 1 so that the bar code reader 33 can read the test tube's bar code 3, irrespective of the rotational position of the test tube 1 when it arrives at the station 12. A computer system (not shown) matches the bar code 3 to a patient's records stored in a database.

After the bar code 3 is read, the test tube 1 is transported to the orientation station 13 by a pushing mechanism 20. The pushing mechanism 20 comprises an endless belt 39 supported by a pulley 40 at each end of the sorting apparatus 10. A plurality of protrusions 41 are attached to the outside of the belt 39, and are equally spaced apart along the belt 39. The belt 39 is driven intermittently by a stepper motor 42 (refer to FIG. 2) rotating one of the pulleys 40 in a direction such that the protrusions 41 on the top side of the belt 39 move in the direction of travel 8.

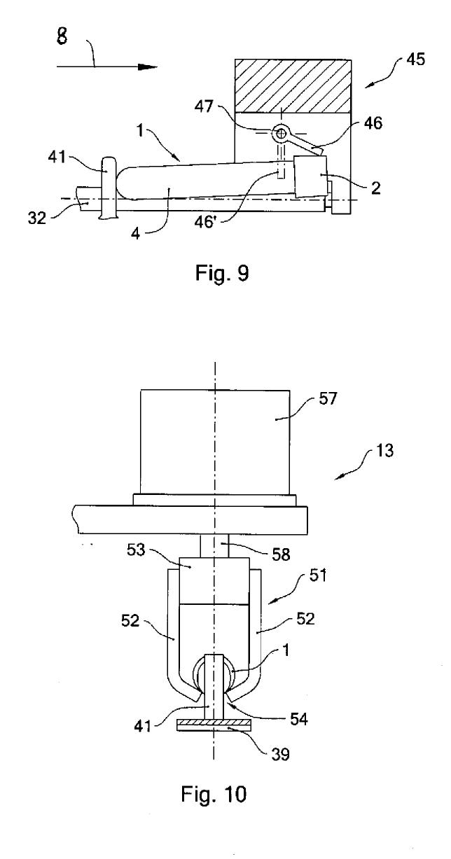

Referring to FIG. 5, as the belt 39 is driven, a protrusion 41 extends into the opening between the rollers 32 to contact an end of the supported test tube 1, and as the protrusion 41 passes through the opening it pushes the contacted test tube 1 to the adjacent orientation station 13. As the test tube 1 is pushed from the bar code reader station 12 to the orientation station 13 it passes a transducer 45. Referring to FIG. 9, the transducer comprises an arm 46 attached to a shaft 47. The arm 46 pivots about the axis of the shaft 47 as indicated by the dashed outline 46′. The shaft 47 is attached to an angular encoder 48 (refer to FIG. 5) such that the inclination of the arm 46 can be measured. As the test tube 1 passes the transducer 45 it contacts the arm 46 thus pivoting it.

The position of the belt 39, and thus the position of the protrusions 41 attached to it, can be determined at any instant from the rotational position of the stepper motor 42. As the test tube 1 passes the transducer 45, the instantaneous inclination of the arm 46 is captured by the encoder 48 at a number of positions of the belt 39. This data is used to determine the orientation and other properties of the passing test tube 1. The cap 2 of the test tube 1 has a larger diameter than the body 4 of the test tube 1, and thus the arm 46 pivots further upwards as the cap 2 passes it than when the body 4 of the test tube 1 passes it. Therefore, the orientation can readily be determined by determining if the cap 2 passes the transducer 45 before or after the body 4 of the test tube 1. Similarly, the captured data can be used to determine the diameter and length of the test tube 1 passing the transducer 45. The length of the test tube 1 can be determined by determining the position of the belt 39 when the test tube 1 first contacts the arm 46, and the diameter can be determined from the arm 46 position as it passes the body 4 of the test tube 1. The length and diameter of each test tube 1 is stored by a computer for later reference.

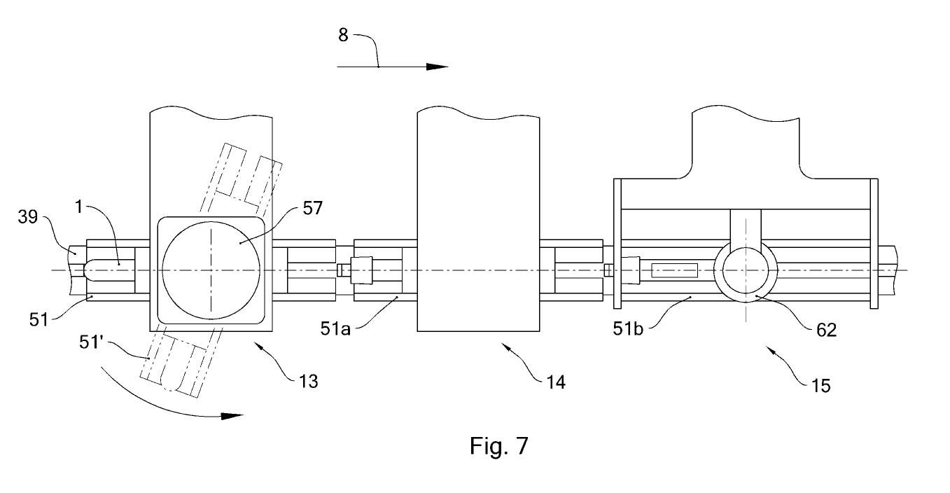

Referring to FIGS. 7, 8 and 10, the orientation station 13 comprises a support device 51. The support device 51 is made up of two side members 52 separated by a block 53. The lower portion of each side member 52 is bent inwards as shown in FIG. 10. The construction of the support device 51 is such that it can carry a test tube 1 on its side whilst providing a recess taking the form of an opening 54 that extends the length of the support device 51. The protrusions 41 of the pushing mechanism 20 extend into and pass through the opening 54 to push a test tube 1 from the orientation station 13 to the adjacent weighing station 14. Preferably, the side members 52 are made from transparent plastic sheet (e.g., polycarbonate).

The orientation station 13 further comprises a rotary actuator 57, such as a stepper motor, that rotates a shaft 58 through angular increments of 180°. The support device 51 is attached to the end of the shaft 58. If a test tube 1 is transported to the orientation station 13 in the wrong orientation with its cap 2 facing the direction of travel 8, as determined by the transducer 45, then the actuator 57 rotates the support device 51 through 180°, as indicated by the dashed outline 51′ in FIG. 7. In this manner, test tubes 1 always leave the orientation station 13 with their body 4 facing the direction of travel 8.

The pushing mechanism 20 transports each test tube 1 from the orientation station 13 to the support device 51a of the weighing station 14. The support device 51a is similar to the support device 51 of the orientation station 13, and is attached to a load cell 60. The load cell 60 measures the weight of the supported test tube 1. The weight may be used to determine the amount of blood in the test tube 1, and is stored by a computer for later reference.

In one embodiment, the amount of blood can be determined by taking the total weight of the test tube 1, subtracting the known tare weight of the test tube 1, and calculating the amount of blood from the weight of the blood. If the tare weight of the test tube 1 is not known, it can in one embodiment be accessed from a look-up table provided by a computer database once the type of test tube has been identified by the transducer 45.

Each test tube 1 is then transported from the weighing station 14 to the support device 51b of the adjacent imaging station 15 by the pushing mechanism 20. The imaging station 15 comprises an image capture device 62, such as a digital camera. The support device 51b is similar to the support device 51 of the orientation station 13 except that it is fully open at the top to provide the image capture device 62 with an unobstructed view of the supported test tube 1. The captured image is analyzed to determine the color of the test tube cap 2, and may also be used to determine other properties of a test tube 1 or blood therein. This information is stored by a computer for later reference.

Each test tube 1 is then transported from the imaging station 15 to the support device 51c of the adjacent transfer station 16 by the pushing mechanism 20. The transfer station 16 transfers each test tube 1 to one of the output locations 17, 18 or 19 (refer to FIG. 2). The output location for an individual test tube 1 is selected based on its bar code 3 as read by the bar code reader station 12. The output location may also be selected based on the dimensions of the test tube 1 as determined by the transducer 45, its weight as measured by weighing station 14, the color of its cap 2 as determined by the imaging station 15 or by the volume of blood in the test tube as calculated above.

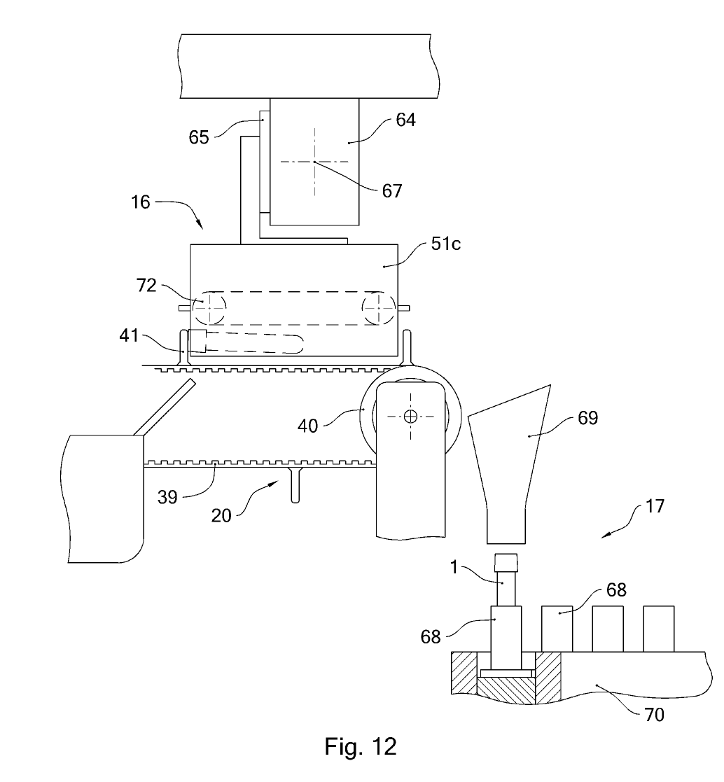

The transfer station 16 comprises a linear actuator 64. Referring to FIG. 12, the linear actuator 64 has a carriage 65, and the support device 51c of the transfer station 16 is attached to the carriage 65. The support device 51c is similar to the support device 51 of the orientation station 13. The carriage 65 is driven by a stepper motor 66 (refer to FIG. 2) through a ball screw or belt drive mechanism (not shown) such that the carriage 65 and the attached support device 51c are movable to any position along an axis 67 that is transverse to the direction of travel 8.

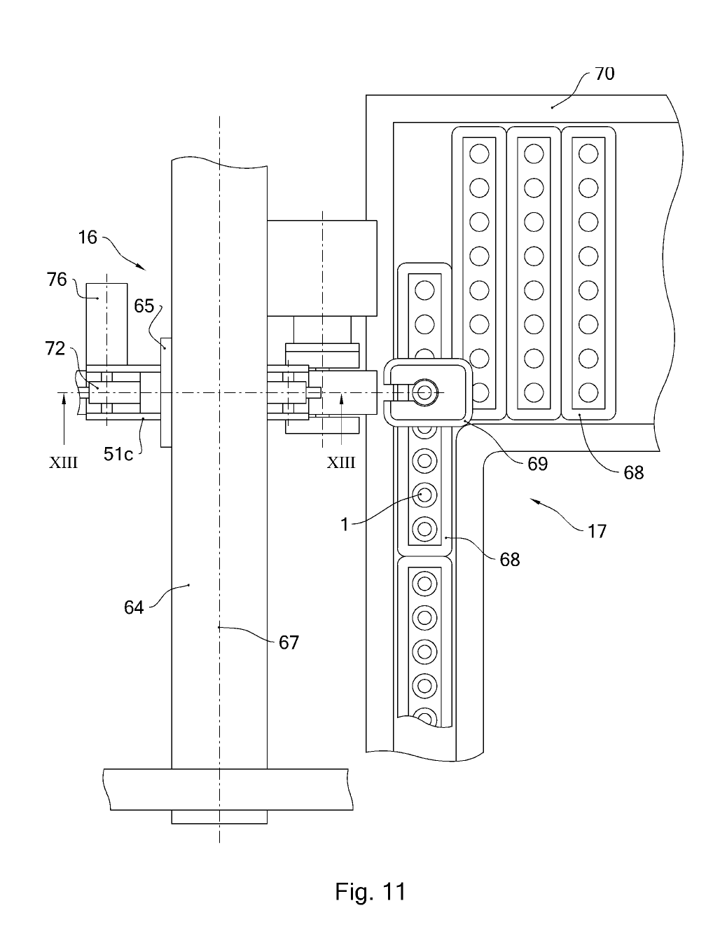

FIGS. 11 and 12 show the transverse position of the support device 51c when a test tube 1 is transported into it. Output location 17 comprises a funnel like guide 69 and a series of one dimensional test tube racks 68. Empty racks 68 are progressively fed along a conveyor 70 and under the guide 69 such that each empty position of each rack 68 aligns in turn with the guide 69. If a test tube 1 at the transfer station 16 is required to be transferred to the racks 68 then the support device 51c remains in the position shown in FIGS. 11 and 12, and as a protrusion 41 of the pushing mechanism 20 passes by it pushes the test tube 1 through the support device 51c and into the guide 69. The test tube 1 then falls through the guide 69 into an empty position of a rack 68. The filled racks 68 can then be manually or automatically transferred to another apparatus to perform the required operations on the test tubes 1.

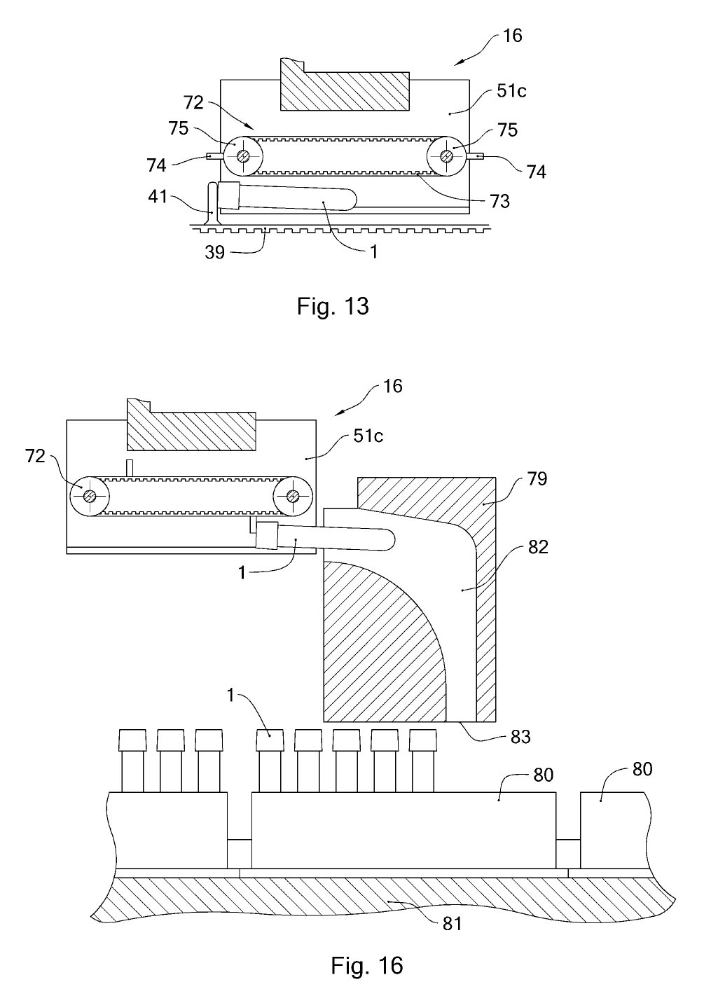

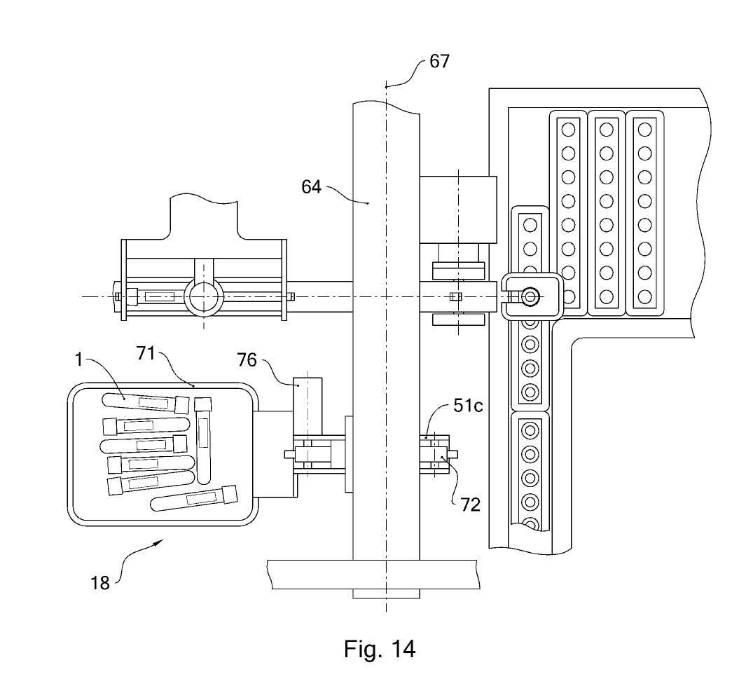

FIG. 14 shows the transverse position of the support device 51c when a test tube 1 is required to be transferred to the output location 18, which is an open container 71. After the test tube 1 is pushed into the support device 51c in the position shown in FIG. 11, the stepper motor 66 transversely moves the support device 51c to the position shown in FIG. 14. Referring to FIG. 13, the transfer station 16 comprises a second pushing mechanism 72 built into the support device 51c. This second pushing mechanism 72 is similar in operation to the main pushing mechanism 20. It comprises a belt 73 supported by two pulleys 75 with two equally spaced apart protrusions 74 attached to the outside of the belt 73. A stepper motor 76 (refer to FIG. 14) can rotate in either direction to move the protrusions 74 forwards or backwards as required. When the stepper motor 76 rotates, the protrusions 74 contact the end of a supported test tube 1, which pushes it out of the support device 51c. After the support device 51c moves to the position shown in FIG. 14, the stepper motor 76 drives the second pushing mechanism 72 such that it pushes the supported test tube 1 backwards out of the support device 51c and into the container 71. Similarly to the racks 68, the container 71 can be manually or automatically transferred if required to perform other operations on the test tubes 1 contained in it.

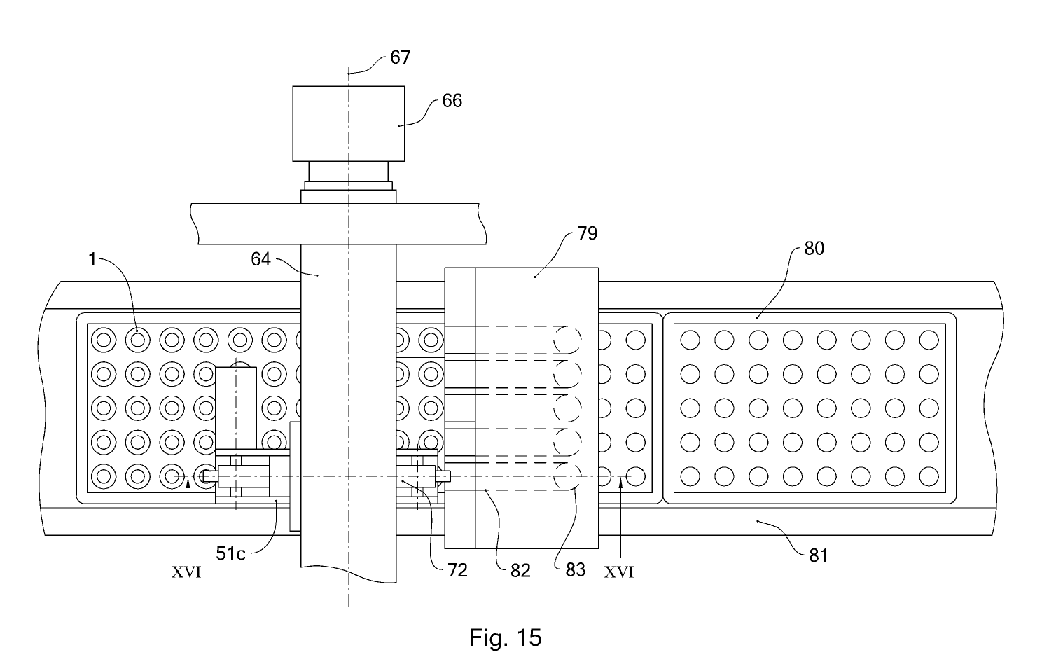

FIGS. 15 and 16 show the transverse position of the support device 51c when a test tube 1 is required to be transferred to the output location 19. Output location 19 comprises a guide block 79 and a series of two dimensional test tube racks 80. In this embodiment, each rack 80 can hold forty test tubes 1 arranged as eight rows of five test tubes 1 per row. The guide block 79 has five holes 82. Each hole 82 is adapted to guide a test tube 1 from the support device 51c to a position in a rack 80. The five holes 82 are arranged such that their exits 83 simultaneously align with five positions on a single row of the rack 80. Empty racks 80 are progressively fed by a conveyor 81 such that each row of each rack 80 aligns in turn with the exits 83 of the guide block 79.

The stepper motor 66 can transversely move the support device 51c to align with any one of the five holes 82. When a test tube 1 is required to be transferred to a rack 80, the support device 51c is positioned to align with one of the holes 82 as shown in FIG. 15, and the second pushing device 72 pushes the supported test tube 1 into the hole 82 as shown in FIG. 16. The test tube 1 then falls through the exit 83 of the hole 82 into a free position in the rack 80. The filled racks 80 can then be manually or automatically transferred to another apparatus to perform the required operations on the test tubes 1.

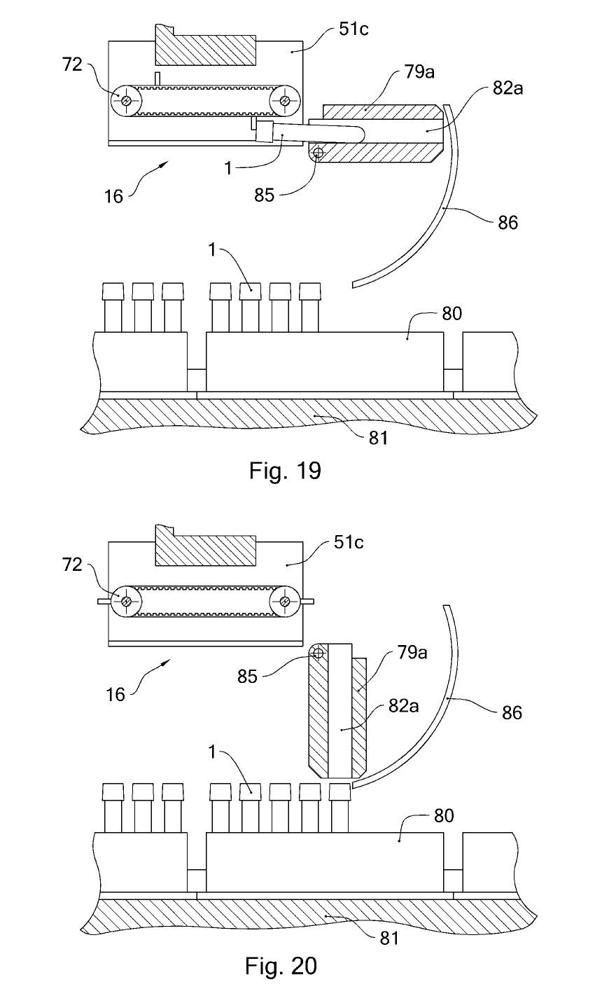

FIGS. 19 and 20 show an alternative guide block 79a for transferring test tubes 1 to a two dimensional test tube rack 80. The guide block has five holes 82a that are spaced apart transversely to align with a row of positions in a rack 80, in a similar manner to the holes 82 in guide block 79. The guide block 79a pivots about a shaft 85. Test tubes 1 are transferred to the guide block 79a with the guide block 79a pivoted upwards such that the holes 82a are horizontal, as shown in FIG. 19. Once a test tube 1 is transferred to each of the holes 82a, the guide block 79a is pivoted downwards and the test tubes 1 fall through the holes 82a into an empty row in a rack 80. A curved barrier 86 prevents the test tubes 1 from falling through the holes 82a until the guide block 79a is pivoted fully downwards.

The sorting apparatus 10 operates by repeatedly driving the pushing mechanism 20 then stopping it whilst the stations 12 to 16 perform their respective functions simultaneously. In this embodiment, each time the pushing mechanism 20 is driven, the protrusions 41 move a distance equal to their spacing, and each time the pushing mechanism 20 stops it remains stationary for enough time to allow the slowest of the stations 12 to 16 to perform its respective function. The throughput in test tubes per hour of the sorting apparatus 10 is relatively high because each station 12 to 16 is dedicated to only a single function, and the stations all operate simultaneously. The design of the transfer station 16 in particular operates rapidly compared to most conventional robotic arms. The overall construction of the apparatus 10 is relatively simple, making the apparatus 10 economical to construct compared with other apparatus that use conventional robotics.

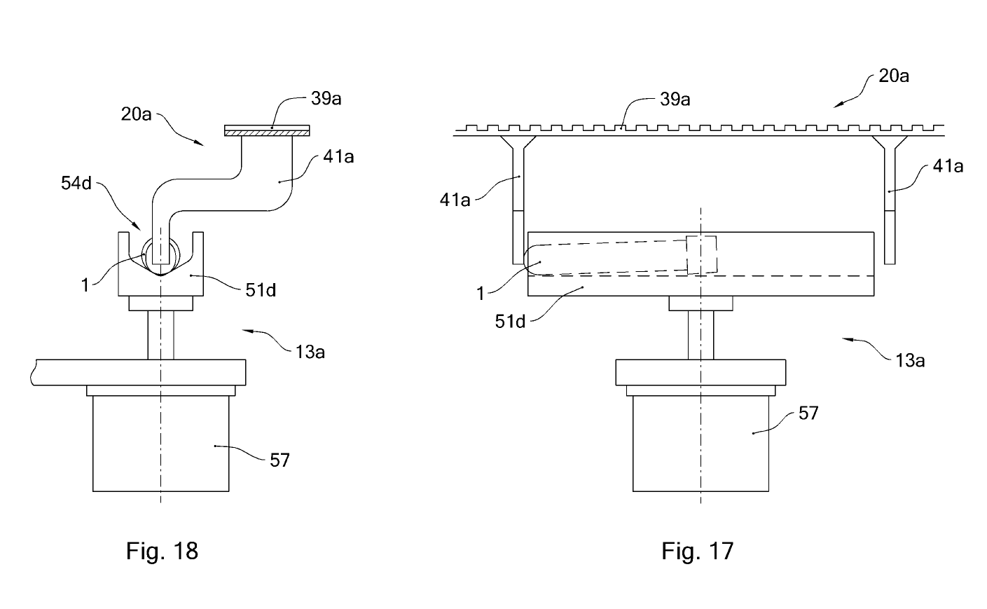

Many different arrangements of support devices and pushing mechanisms are possible within the scope of the invention. As an example, FIGS. 17 and 18 show an alternative embodiment of an orientation station 13a having an alternative support device 51d. Support device 51d has an open channel like construction. It supports a test tube 1 on its side like support device 51, but it has a recess in the form of opening 54d extending its length above the supported test tube 1. In this embodiment, an alternative pushing mechanism 20a comprises a belt 39a running above and to the side of the orientation station 13a with protrusions 41a extending down into the opening 54d to push the test tube 1.

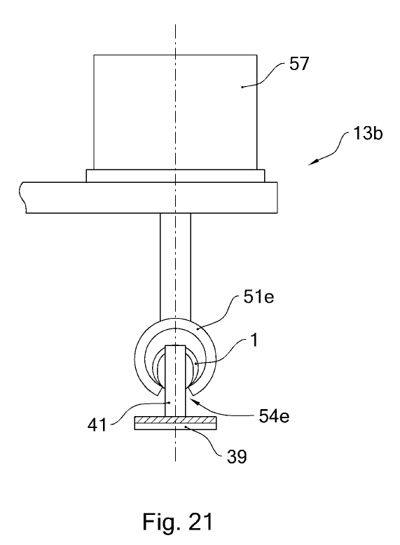

FIG. 21 shows a further alternative embodiment of an orientation station 13b having a further alternative support device 51e for a test tube sorting apparatus in accordance with the present invention. The support device 51e is constructed from a tube having a recess along its length in the form of an opening 54e.

The embodiments described above have pushing mechanisms comprising a single endless belt 39. However, in other embodiments two or more belts may be used in sequence, each with protrusions 41 attached. Furthermore, the pushing protrusions 41 may be attached to and driven by means other than an endless belt.

In other embodiments the transducer 45 may comprise an arm 46 that moves linearly, rather than pivoting, or in still other embodiments the transducer 45 may comprises a non-contact sensor such as a laser beam.

The scope of the present invention is not limited to the arrangement of the sorting apparatus 10 described above. Alternative embodiments of the invention may have more or less stations, and more or less output locations than the sorting apparatus 10. For example, a sorting apparatus may not have a weighing or imaging station. Or, an embodiment of the present invention may for example only have a single output location. Also, it is possible for two functions to be combined at a single station. For example, an image capture device may be added to the bar code reader station or the image capture device may be used to read the barcode as well, or a load cell may be built into the orientation station.

While the invention has been described with reference to a number of preferred embodiments it should be appreciated that the invention can be embodied in many other forms. For example, each support device could include a cradle, the recess being provided by an open top of the cradle and extending along the length of the cradle. The protrusions would pass through the open top of the cradle in use to push the test tubes. In this case, the endless conveyor would be located above the support devices. The structural form of the cradle can take many different forms, for example, it could take the form of a solid body or a structural framework.

In yet another embodiment, each support device could include a C-type section oriented to provide a side recess extending along the length of the C-type section. The protrusions would pass through the side recess of the C-type section in use to push the test tubes. In this case, the endless conveyor would be located to one side of the support device. The structural form of the C-type section can take many different forms, for example, it could take the form of a solid body or a structural framework.

It should be understood that although the detailed description of the invention refers to transporting and sorting test tubes, this is but one example of a type of vessel that can be transported and/or sorted by embodiments of the invention. For example, other embodiments of the invention are adapted for transporting and/or sorting elongated vessels, bowls, bottles, containers and/or beakers. The vessel may be empty or may contain a substance (e.g., blood).

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

In the claims which follow and in the preceding description of the invention, except where the context requires otherwise due to express language or necessary implication, the word “comprise” or variations such as “comprises” or “comprising” are used in an inclusive sense, i.e., to specify the presence of the stated features but not to preclude the presence or addition of further features in various embodiments of the invention.

1. An apparatus for transporting and sorting a vessel, comprising:
at least two stations with a respective support device, at least one of the support devices operable to support the vessel in a reclined orientation;
a pushing mechanism adapted to transport the vessel along the support devices from one of the at least two stations to another of the at least two stations, the pushing mechanism comprising a plurality of protrusions, each protrusion operable:
to move between the at least two stations; and
to contact a vessel supported at one of the at least two stations and transport the vessel to another of the at least two stations;
each of the support devices having a recess extending along a length thereof through which the protrusions pass in use;
at least one of the at least two stations operable to acquire information relating to the vessel; and
at least another of the at least two stations being a transfer station operable to transfer the vessel to one of at least two output locations dependent upon the information acquired, thereby sorting the vessel between the at least two output locations.

2. An apparatus according to claim 1, further comprising a transducer operable to determine an orientation of the vessel.

3. (canceled)

4. An apparatus according to claim 1, wherein at least one of the support devices is rotatable to change an orientation of the vessel.

5. An apparatus according to claim 1, wherein:
the vessel is elongate and has at least one end;
at least one of the support devices is operable to support the vessel in lengthwise alignment; and
each of the protrusions is operable to contact the at least one end of the vessel to push the vessel lengthwise along at least one of the support devices.

6. An apparatus according to claim 5, wherein the at least one support device is operable to support the vessel in lengthwise alignment.

7. An apparatus according to claim 6, further comprising a transducer adapted to determine the orientation of the vessel.

8. An apparatus according to claim 7, wherein the transducer comprises an arm that contacts the vessel as it is transported past the transducer.

9. An apparatus according to claim 8, wherein the orientation of the vessel is determined by measuring the position of the arm and the longitudinal position of the vessel as it is transported past the transducer.

10. An apparatus according to claim 8, wherein the arm of the transducer is mounted on a pivot.

11. An apparatus according to claim 7, wherein the transducer is operable to measure the length of the vessel.

12. An apparatus according to claim 7, wherein the vessel has a round cross-section and the transducer is further adapted to measure the diameter of the vessel.

13. (canceled)

14. An apparatus according to claim 1, wherein the protrusions are attached to an endless conveyor in spaced apart relation.

15. (canceled)

16. An apparatus according to claim 1, wherein:
a vessel is supported by the support device; and
the recess in each support device is below the vessel supported by the support device in use.

17. An apparatus according to claim 1, wherein:
the vessel travels in a main direction through the at least two stations;
the support device of the transfer station is movable in a direction that is transverse to the main direction.

18. An apparatus according to claim 17, wherein the transfer station comprises a second pushing mechanism operable to push a vessel out of the transfer station.

19. An apparatus according to claim 1, wherein at least one of the output locations is a vessel rack.

20. An apparatus according to claim 1, wherein at least one of the at least two stations comprises a bar code reader operable to read a bar code on a vessel.

21. An apparatus according to claim 20, wherein the vessel is elongate and the support device of the station comprising the bar code reader comprises a pair of rollers adapted to rotate a vessel on its longitudinal axis.

22. An apparatus according to claim 20, further comprising a database operatively connected to the bar code reader and operable to match the bar code of a vessel to a patient.

23. An apparatus according to 1, further comprising a feeder operable to fee vessels to at least one of the support devices.

24. An apparatus according to claim 23, wherein the feeder comprises an outlet, a hopper for receiving the vessels, and a depositor operable to receive the vessels from the outlet of the hopper and sequentially deposit the vessels on at least one of the support devices.

25. An apparatus according to claim 1, wherein at least one of the at least two stations is operable to weigh a vessel.

26. An apparatus according to claim 1, wherein at least one of the at least two stations comprises an image capture device.

27. An apparatus according to claim 26, wherein:
the vessel has a cap; and
the image capture device is operable to detect the color of the cap.

28. An apparatus according to claim 1, wherein the vessel is a test tube.

29. An apparatus according to claim 28, wherein the test tube is operable to store a blood sample.

30. (canceled)

31. A method of sorting vessels comprising the following steps:
i. Depositing a vessel in a feeder;
ii. Feeding the vessel to a support device of a station where the vessel is supported on a side thereof in a reclined orientation;
iii. Transporting the vessel, while in the reclined orientation, to another station;
iv. Acquiring information relating to the vessel;
v. Transporting the vessel to another station; and
vi. Transferring the vessel to one of two or more output locations dependent upon the information acquired.

32. A method of sorting vessels as defined in claim 31, wherein the vessel is a test tube.

33. (canceled)

34. (canceled)

35. (canceled)

36. (canceled)

37. A method of sorting vessels according to claim 31, which further comprises repeating steps iv and v at least once before progressing to step vi.
